(19) United States
(12) Patent Application Publication
(10) Pub. No.: US 2013/0005972 A1
(43) Pub. Date: Jan. 3, 2013

(54) Application of Staudinger Ligation in PET Imaging

(75) Inventors: Dhanalakshmi Kasi, Los Angeles, CA (US); Umesh Gangadharmath, Los Angeles, CA (US); Joseph Walsh, Pacific Palisades, CA (US); Hartmuth Kolb, Playa Del Rey, CA (US)

(73) Assignee: SIEMENS MEDICAL SOLUTIONS USA, INC., Malvern, PA (US)

(21) Appl. No.: 13/634,308

(22) PCT Filed: Mar. 29, 2011
(86) PCT No.: PCT/US11/30325

§ 371 Date: Sep. 12, 2012

Related U.S. Application Data
(60) Provisional application No. 61/318,519, filed on Mar. 29, 2010.

(51) Int. Cl. C07C 231/10; C07C 269/06; C07D 239/95; C07D 233/91; C07D 403/06

(52) U.S. Cl. 544/287; 564/183; 548/327.5; 548/255; 560/31

(57) ABSTRACT

A method for generating a radiolabeled tracer. The method includes providing a phosphine molecule having at least one carbocyclic, aromatic, or pyrrolidinyl ring with an OH substitute. The OH of this phosphines molecule is then condensed with an acid to produce a phosphine ester. Staudinger ligation is then performed to generate the radiolabeled tracer by treating the phosphine ester with a radiolabeled azide having a PET radioisotope moiety.

This application is the U.S. national phase application of PCT International Application No. PCT/US2011/030325, filed on Mar. 29, 2011, which claims priority to U.S. Provisional Patent Application No. 61/318,519 filed on Mar. 29, 2010, the contents of such applications being incorporated by reference herein.

### BACKGROUND OF THE INVENTION

1. Field of the Invention

The present invention relates to the field of Positron Emission Tomography (PET). In particular, it relates to a method of synthesizing radiolabeled molecules, which can be detected with PET. The present invention also provides novel compounds that may be used in the various methods for synthesizing a radiolabeled molecule.

2. Background of the Invention & Description of Related Art

Positron emission tomography (PET) is a diagnostic imaging technique for measuring the metabolic activity of cells in vivo. For example, PET can show images of glucose metabolism in the brain and rapid changes in activity at various time points. It can be used to show changes in physiology before any change in gross anatomy has occurred. PET has been used in detecting diseases such as cancer, heart disease, Alzheimer's disease, Parkinson's disease, and schizophrenia.

PET uses chemical compounds that are labeled with radioactive atoms that decay by emitting positrons. The most commonly used PET radioisotopes are ¹¹C, ¹³N, ¹⁵O, and ¹⁸F. Typically, the labeled compound is a natural substrate, substrate analog, or drug that is labeled with a radioisotope without altering the compound's chemical or biological properties. After injection into an animal, the radiolabeled compound should follow the normal metabolic pathway of its unlabeled analog. The labeled compound emits positrons as it moves through tissues. Collisions between the positrons and electrons that are present in the tissue emit gamma rays that are detectable by a PET scanner.

Construction of radiolabeled tracers by direct labeling involves the coupling of both an activated labeling species, such as ¹⁸F-fluoride, and a highly activated precursor molecule. Subsequent steps may be required to further elaborate the initial radiolabeled species into the final imaging agent. For example, preparation of ¹⁸F-FLT is accomplished by reacting ¹⁸F-fluoride with a protected nosylated precursor, which after labeling, is deprotected and purified to generate ¹⁸F-FLT suitable for PET imaging. This example illustrates the many drawbacks associated with this method of tracer labeling. First, the very nature of labeling with ¹⁸F-fluoride severely limits the choice of labeling precursors. Typical precursors suitable for direct labeling include the use of activated alkyl and/or aryl sulfonate esters. Secondly, acidic protons cannot be present in the labeling precursor or the labeling efficiency will suffer. This specific limitation prevents the direct labeling of compounds containing moieties such as free carboxylic acids, ammonium salts, sulfonamides and sometimes even amides. Finally, direct labeling requires high temperatures which can lead to excessive decomposition of both the precursor and product leading to purification difficulties and inefficient labeling. There is a great need for labeling protocols that are synthetically easy to perform under milder conditions and allow for the synthesis of a broader range of tracers.

A number of milder labeling methods involve the use of click chemistry, oxime condensations, reductive aminations and amide couplings with activated esters (cf. Scheme 1). While these techniques have been successfully employed to label a variety of tracers including peptides and small molecules, each technique introduces additional labeling restrictions.

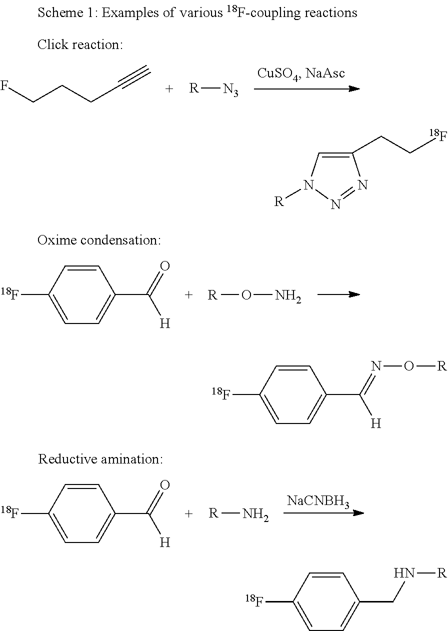

The use of click chemistry for ¹⁸F-labeling for the introduction of a 1,2,3-triazole into the tracer, requires the simultaneous use of both an azide and alkyne coupling partner. Click chemistry is advantageous for radiolabeling since the coupling occurs quickly, cleanly and tolerates a wide array of solvents, including water.

The use of oxime condensations typically involves coupling of ¹⁸F-labeled aldehydes with oxy-amino starting materials. Introduction of the oxy-amino group requires several synthetic steps, with the extra issue of the oxy-amino groups themselves degrading over time.

Reductive aminations using either ¹⁸F-labeled aldehydes or ¹⁸F-labeled amines are sensitive to reaction conditions and extensive optimization is often needed in order to obtain high radiochemical conversions. In addition, copious amounts of reducing agents, such as NaCNBH₃, may lead to unwanted side reactions thus decreasing the overall labeling yield.

Finally, amide couplings using ¹⁸F-labeled activated esters have been used extensively for labeling of biomacromolecules. Unfortunately, the preparation of these activated esters requires many steps and leading to a very lengthy labeling protocol. In addition, coupling yields can be severely hampered by the presence of trace amounts of water.

A milder protocol that is less sensitive to the presence of water, results in labeling with high efficiency and is compatible with wide variety of functional groups would be a vast improvement over traditional labeling methods.

The Staudinger reaction, first published in 1919, described the preparation of phosphoazo compounds from phosphine and azide coupling partners via loss of N₂. In the presence of water, the phosphoazo complex generates an amine and a concomitant phosphine oxide. Several variants of the Staudinger reaction are known and often utilized in chemistry including the formation of amines from azides, aziridines from alpha-hydroxy azides and amides from phosphine-containing esters. The latter reaction is commonly referred to as the Staudinger ligation.

The Staudinger Ligation, an amide bond forming reaction between an azide and a phosphine containing ester, was developed by Saxon et al, “A ‘Traceless’ Staudinger Ligation for Chemoselective Synthesis of Amide Bonds”, Organic Letters, American Chemical Society, vol. 2, no. 14, pp. 2141-2143, (Jun. 20, 2000). In this reaction, the amide linkage was created by a chemoselective ligation between an azide and a triaryl phosphine. The mechanism of the reaction involves nucleophilic attack of a phosphine on an azide to form a phosphazide, which after loss of nitrogen and hydrolysis with water results in the formation of a phosphine oxide and amide. This reaction possesses several advantages over conventional amide coupling reactions that employ amines and activated acid derivatives. In conventional coupling methods, coupling yields are usually low due to competing side reactions such as the hydrolysis of activated esters by adventitious water. Additionally, racemization of the substrates and/or products can be a major problem in conventional amide coupling reactions when chiral centers are present. The Staudinger ligation does not suffer from these limitations. For example, the Staudinger ligation is widely used in carbohydrate chemistry to avoid racemization issues.

The Staudinger ligation has been used successfully for several applications including cell surface engineering, probing post-translational modifications of proteins and for coating microarrays. This reaction is highly selective and can be carried out on biomolecules in an aqueous medium, even on the surface of living cells.

Saxon et al. designed a triarylphosphine containing an aryl group which is functionalized by an ester adjacent to the phosphorous (cf. Scheme 2). The proximity of the functional groups helps to facilitate the intramolecular trapping of the aza glide intermediate and followed by an acyl transfer step.

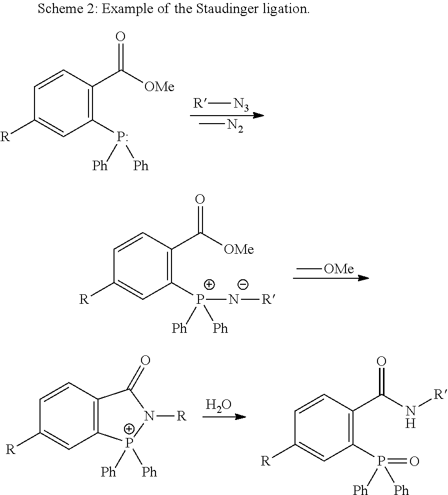

The aza-ylide reacts preferentially to the adjacent carbonyl, via a nucleophilic attack of the nitrogen atom onto the ester, during the hydrolysis step. This transformation was so efficient and mild that it was executed on the surface of living cells. One limitation of this method is the automatic incorporation of the bulky arylphosphine as part of the amide construct. As a consequence, any compound produced by using this method must contain a triarylphosphine oxide moiety in the final product. The presence of this phosphine oxide may not be suitable for preparing glycoconjugate vaccines or even small molecules, as the presence of the phosphine oxide could significantly deter substrate binding.

In an effort to avoid the problem of having a disruptive triarylphosphine oxide moiety, two groups (i.e., Saxon et al., and Nilsson et al., “High-Yielding Staudinger Ligation of a Phohinothioester and Azide to Form a Peptide”, Organic Letters, American Chemical Society, vol. 3, no. 1, pp. 9-10, (Dec. 19, 2000)) have independently prepared a new generation of phosphines which are released from the conjugated product, thus inventing the traceless Staudinger ligation (Scheme 3). In this method, the final product is an amide without the phosphine oxide present, thus allowing broader use of this reaction. In this new generation, the phosphine is part of the leaving group and not conjugated to the transferred acyl group. In the example given by Saxon et al., the t_{1/2} for the ligation reaction was reported to be 18 hours. This long half-life is not compatible for labeling with short-lived isotopes such as ¹⁸F-fluorine. In the example given by Nilson et al., the t_{1/2} for the ligation reaction was reported to be several hours for both the oxygen and thio-phosphine derivatives. Again, this prolonged reaction kinetics makes the use of these ligands incompatible for ¹⁸F-labeling.

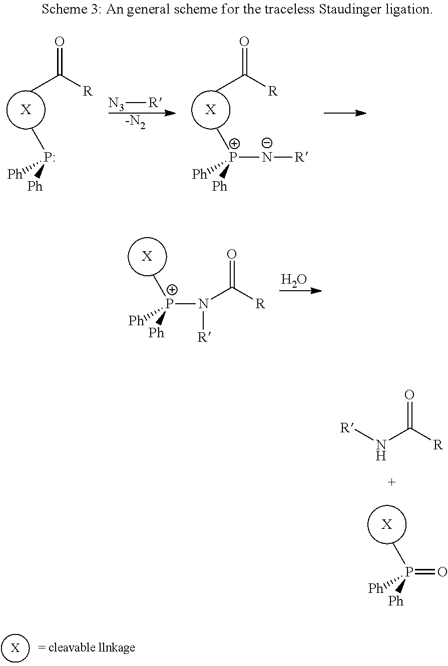

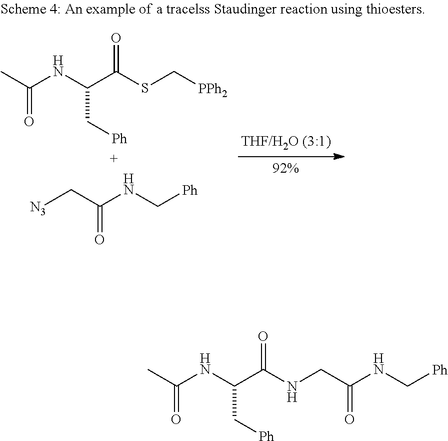

Raines et al, “Reaction Mechanism and Kinetics of the Traceless Staudinger Ligation”, J. Am. Chem. Sc., vol. 128, no. 27, pp. 8820-8828 (Jun. 20, 2006), compared the reactivity of various phosphine ligands for the Staudinger Ligation, shown below. They observed that the phosphines 1 and 5 have similar reactivity and afforded amides as the exclusive product. No amine by-product was observed when using phosphine 5, but some amine by-product was observed when using phosphine 1.

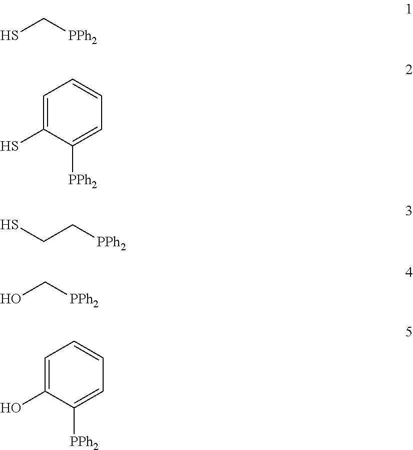

Despite the excellent reactivity of phosphine 1 and 5 for forming amides, these phosphines contain several inherent limitations. First, the preparation of 1 is lengthy and time consuming. Secondly, the shelf-life of 1 is very short owing to rapid air oxidation. Phosphine 5 is a more suitable moiety for Staudinger-based ligations as it appears to have a longer shelf-life. However, both 1 and 5 have long t_{1/2} reaction rates that are not compatible with ¹⁸F-labeling. New phosphines are needed which are easy to prepare, stable and can rapidly perform couplings with fast rates to accommodate the short half-lives of positron emitters. In addition, the ligation chemistry must tolerate a wide range of functional groups and reaction conditions.

### BRIEF DESCRIPTION OF THE DRAWINGS

FIG. 1 shows LCMS data of phosphine 8b.

FIG. 2 shows LCMS Data for Compound 19.

### SUMMARY OF THE INVENTION

A method for generating a radiolabeled tracer, the method comprising: providing a compound of the following Formula I:

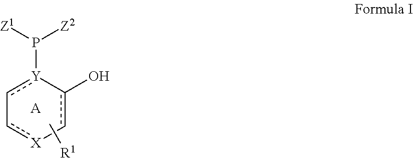

In Formula I, X is C, N, or a bond, and Y is C or N. When X is C, ring A is either aromatic or saturated. When X is N, ring A is aromatic. When X is a bond, ring A is saturated.

R¹ is H, an electron withdrawing group, or an electron donating group.

Z¹ and Z², together with the phosphorus atom to which they are attached to, may form a substituted or unsubstituted, heterocyclic ring. Alternatively, Z¹ and Z² together may be ferrocene, with the P atom being connected to each cyclopentadiene ring. As another alternative, Z¹ and Z² may each independently be carbocyclic, heterocyclic, aryl, heteroaryl, or NR²R³. When either Z¹ or Z² is NR²R³, R² and R³ are each independently H, alkyl, aryl, an electron withdrawing group, or an electron donating group.

When R¹ is H, at least one of Z¹ and Z² is an aryl which includes a substituent which is an electron withdrawing group or an electron donating group.

In a first step of the method for generating a radiolabeled tracer, the OH of Formula I is condensed with an acid to produce a phosphine ester. Staudinger ligation is then performed to generate the radiolabeled tracer by treating the phosphine ester produced in the first step with a radiolabeled azide having a PET radioisotope moiety Q as the radiolabel.

### DETAILED DESCRIPTION OF EMBODIMENTS

It is to be understood that the figures and descriptions of the present invention have been simplified to illustrate elements that are relevant for a clear understanding of the present invention, while eliminating, for purposes of clarity, many other elements which are conventional in this art. Those of ordinary skill in the art will recognize that other elements are desirable for implementing the present invention. However, because such elements are well known in the art, and because they do not facilitate a better understanding of the present invention, a discussion of such elements is not provided herein.

The present invention will now be described in detail on the basis of exemplary embodiments.

The process describes the rapid radiolabeling of the acid via traceless Staudinger ligation to generate amide compounds.

The process involves the generation of radiolabeled tracers from a phosphine with the general formula:
wherein X is C, N, or a bond;

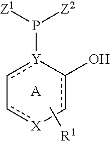

wherein Y is C or N;
wherein:
   ring A is either aromatic or saturated when X is C;
   ring A is aromatic when X is N; and
   ring A is saturated when X is a bond;
wherein R¹ is H, an electron withdrawing group, or an electron donating group;
wherein:
   Z¹ and Z², together with the phosphorus atom to which they are attached to, form a substituted or unsubstituted, heterocyclic ring;
   Z¹ and Z² together are ferrocene with the P atom being connected to each cyclopentadiene ring; or
   Z¹ and Z² are each independently carbocyclic, heterocyclic, aryl, heteroaryl, or NR²R³;
   wherein R² and R³ are each independently H, alkyl, aryl, an electron withdrawing group, or an electron donating group; and
wherein, when R¹ is H, at least one of Z¹ and Z² is an aryl which includes an electron withdrawing group substituent, or an electron donating group substituent.

Electron withdrawing groups include CN, CF₃, F, Cl, Br, COR⁴, CONH₂, SONH₂, SO₃R⁵, and NO₂ with R⁴ and R⁵ each being independently selected as H, alkyl, or aryl.

Electron donating groups include alkyl, aryl, O-alkyl, O-aryl, NH₂, NHR⁶, NR⁷R⁸, and NHCOMe, with R⁶, R⁷, and R⁸ each being independently selected as alkyl or aryl.

Specific examples of such phosphines include, but are not limited to, the following general formulas:

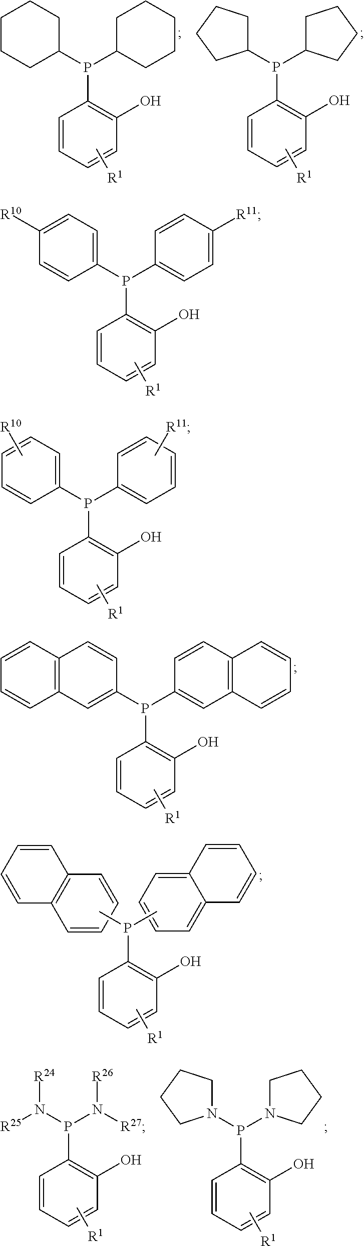

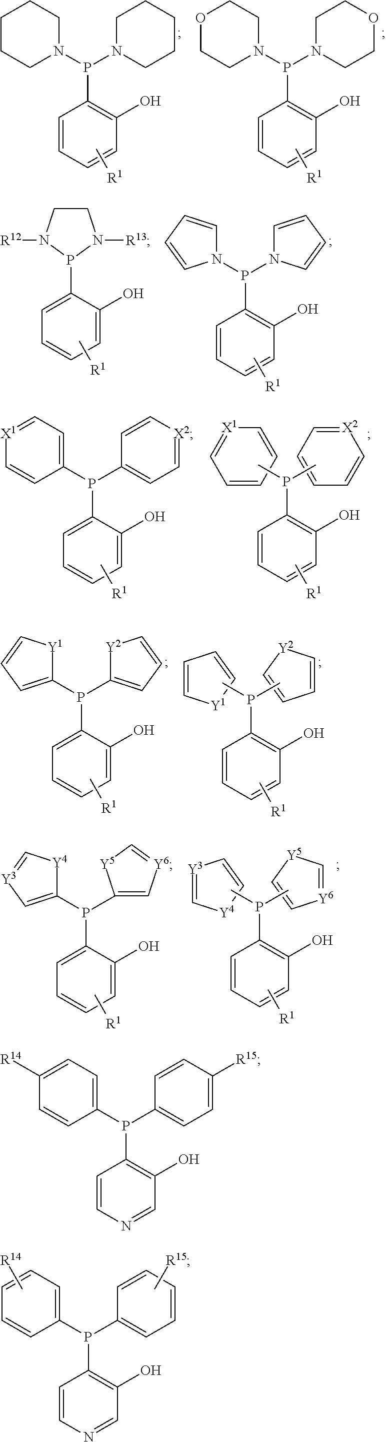

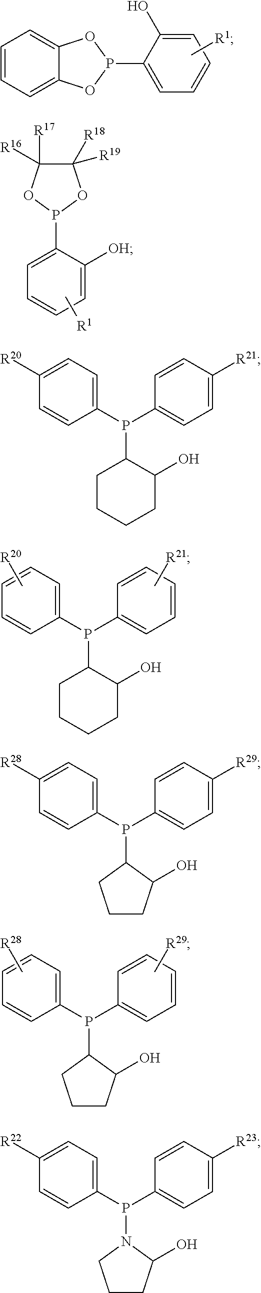

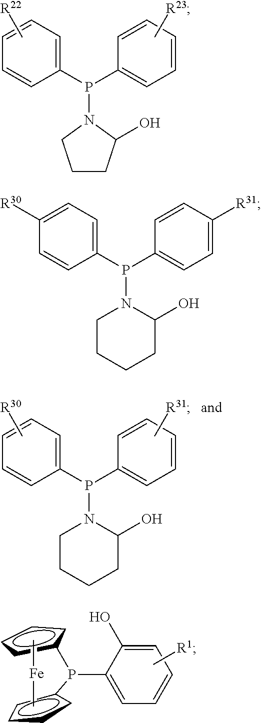

where R¹⁰ to R³¹ are each independently H, alkyl, aryl, an electron withdrawing group, or an electron donating group; where X¹ and X² are each independently CH or N; where Y¹, Y², Y⁴, and Y⁵ are each independently S, O, NH, or CH₂; and where Y³ and Y⁶ are each N or CH.

Any of the above compounds may be used in the reactions of the present invention.

The phenolic OH of the phosphine is condensed with various acids like aliphatic, aromatic, amino heterocycle, heteroaryl of the general formula:

R′—COOH;

where R′=alkyl, aryl, aminoalkyl, sugar, heterocycle, heteroaryl.

The coupling generates the phosphine esters which, in one example, are of the general formula:

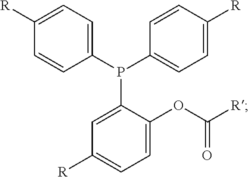

where each R is independently H, alkyl, aryl, an electron withdrawing group, or an electron donating group.

It will be understood that the use of each different compound above will generate different but analogous phosphine esters. For example, using the phosphine with two cyclohexane molecules may generate a slightly different ester from the example above.

The acylated phosphine precursor is then treated with the radiolabeled azide to generate the labeled compound of formula:

Q-B—N₃+R′—CO—OL→Q-B—NH—CO—R′;

where Q is a PET radioisotope moiety; B is defined as alkyl, aryl, aminoalkyl, sugar, heterocycle, or heteroaryl; and the “OL” is the phosphine moiety, with the O of the “OL” coming from the OH group of the original phosphine.

Examples of appropriate PET radioisotope moieties Q for the radiolabeled azide include ¹¹C, ¹³N, ¹⁵O, and ¹⁸F, with ¹⁸F being a particularly suitable radioisotope moiety.

### Examples

Synthesis of Phosphine Ligand:

Diphenylphospine was coupled with iodophenol in the presence of palladium acetate at 100° C. to give the corresponding phosphine phenol. The phenol was benzoylated at room temperature to yield the benzoyl phosphine.

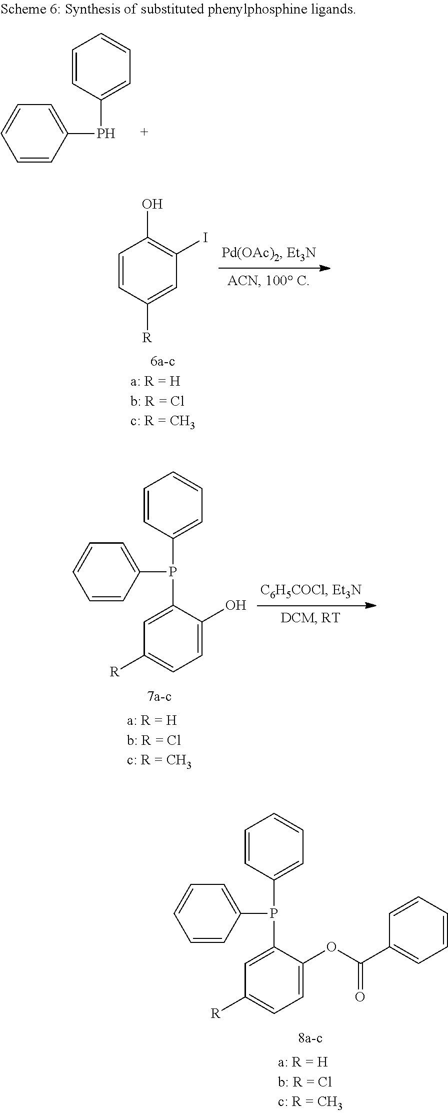

Staudinger Ligation:

The reaction conditions were optimized by changing the solvent, temperature and substituent on the phosphine.

Effect of Temperature:

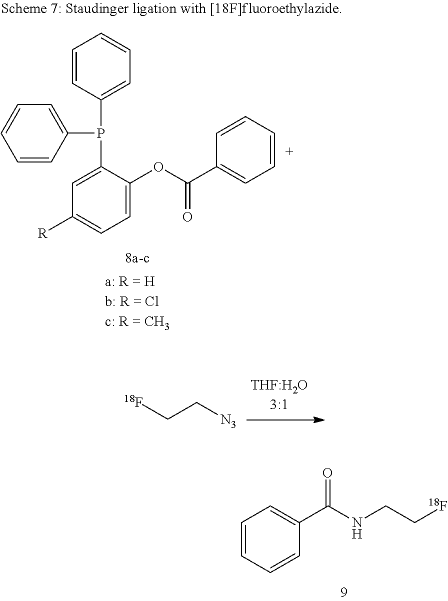

| | | Amide Formation | |
|---|---|---|---|
| | | (%) | |
| Phosphine | R | 80° C. | 100° C. |
| 8a | H | 13 | 26 |
| 8b | Cl | 3 | 8 |
| 8c | CH₃ | 27 | 60 |

The Staudinger ligation was performed by treating the phosphine ester with ¹⁸F-ethylazide. The reaction time was shortened (10 min) to accommodate the rapid the radioactivity decay of ¹⁸F-fluoride. The phosphine as described by Saxon at al. (R═H), poorly converted the phosphine ester into the resultant amide, even at elevated temperatures. The chloro-analog performed very poorly and afforded very little conversion to the desired product. By adding a modestly electron donating group, the conversion to the desired product increased dramatically.

The reaction mixture cleanly afforded the desired production without formation of the unwanted ¹⁸F-fluoroethyl amine. Because the conversion to product is relatively clean, the purification process is relatively simple affording a higher probability of isolating a pure product within a timeframe compatible with the half-life of ¹⁸F-fluorine (t_{1/2}=110 min).

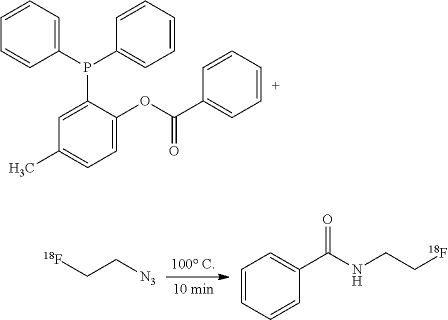 [SMILES: Cc1ccc(OC(=O)c2ccccc2)c(P(c2ccccc2)c2ccccc2)c1 ; [N-]=[N+]=NCC[18F] ; O=C(NCC[18F])c1ccccc1]

Effect of Solvent:

The choice of solvents for the coupling was relevant to the formation of the desired product. Water was relevant for the formation of the amide. Aqueous THF afforded the highest coupling percentage. Addition of DMF, which was reported by Nilson et al. to afford the best coupling yields, hurt the coupling yield. Addition of DMSO also hurt the coupling reaction.

| | | Amide |
|---|---|---|
| Solvent mixture | Ratio | Formation (%) |
| THF:H₂O | 3:1 | 60 |
| DMF:THF:H₂O | 3:1:1 | 37 |
| DMSO:THF:H₂O | 3:1:1 | 38 |
| THF | 100% | 0 |

Stability of the Phosphines:

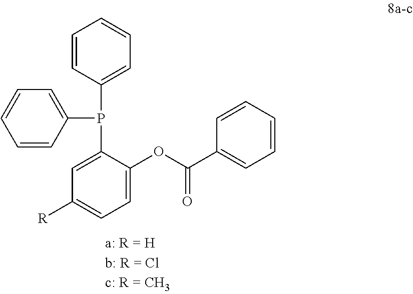

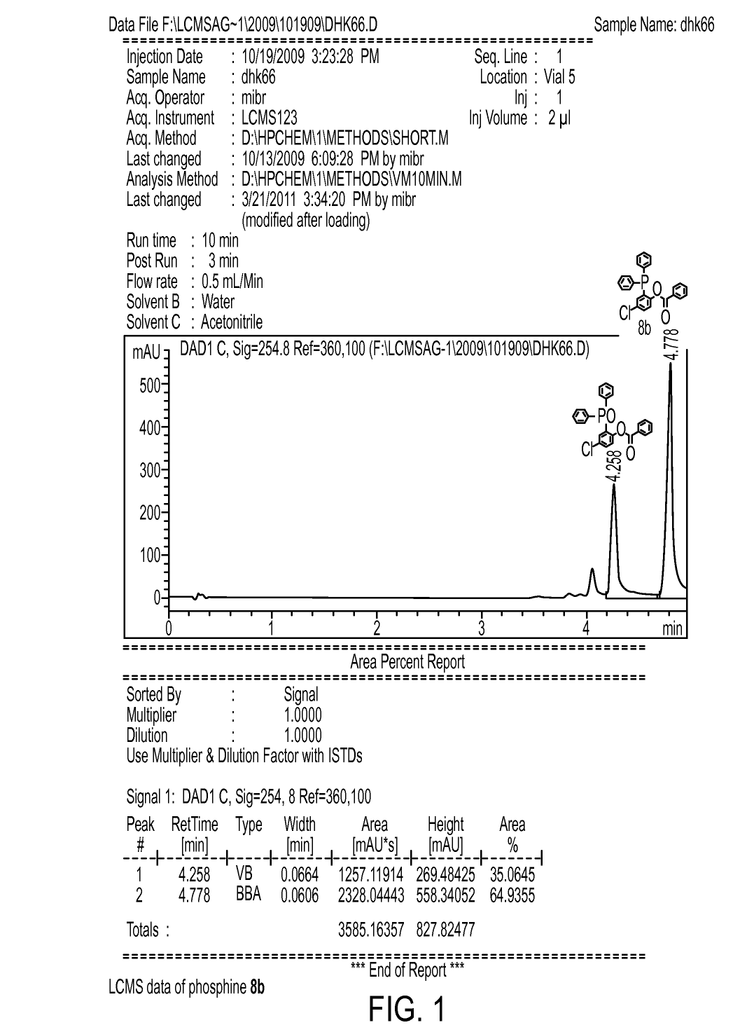

Phosphines 8a and 8c exhibit excellent stability profiles at room temperature, while the chloro compound 8b found to oxidize during the isolation using column chromatography. 35% of oxidized phosphine was formed within an hour at room temperature during the isolation as shown in FIG. 1. Oxidation was minimized by storing the compound under Ar at −20° C.

Synthesis of Heterocyclic Phosphine:

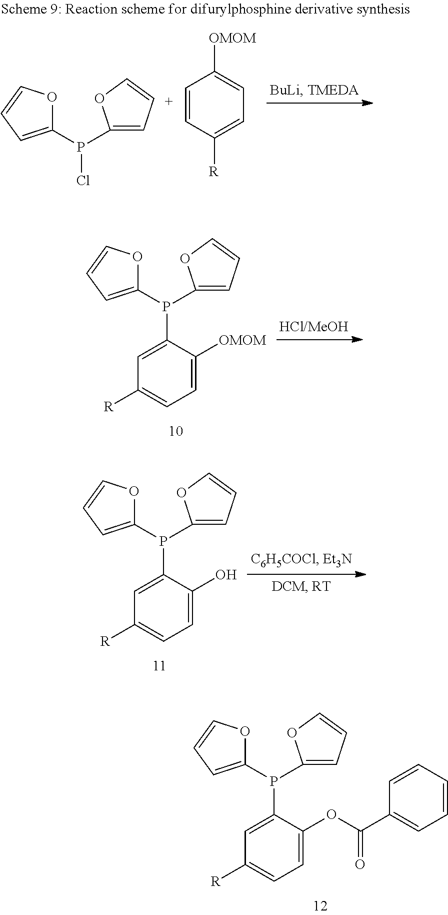

Application of Staudinger Ligation:

1. Application of Staudinger Ligation in Hypoxia Imaging:

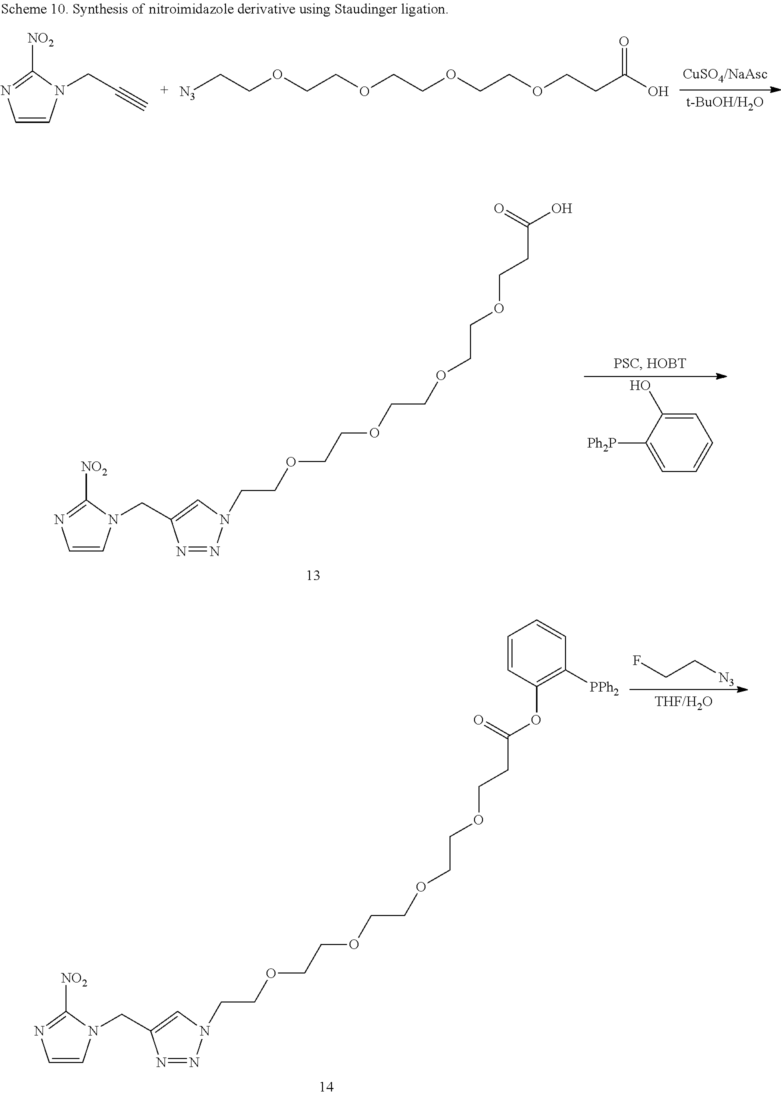

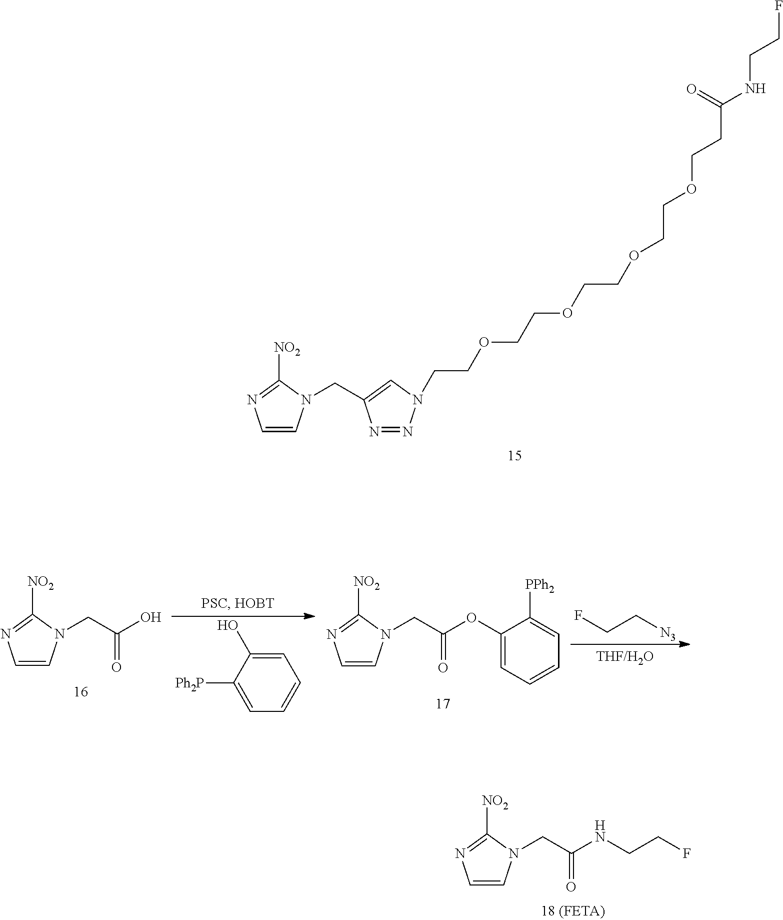

Stability of the Phosphine Esters 14 and 19:

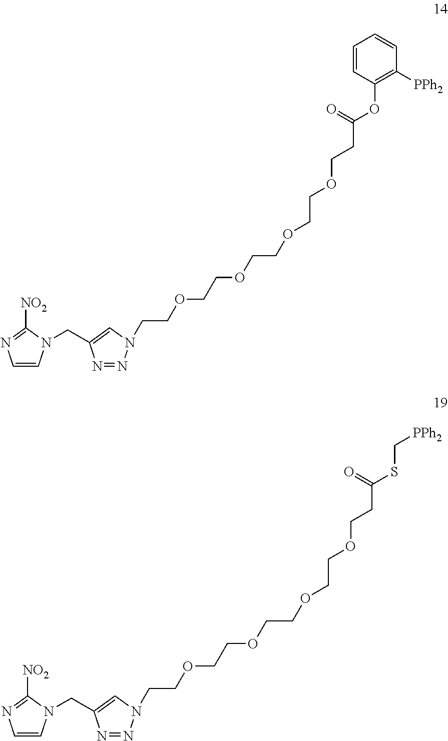

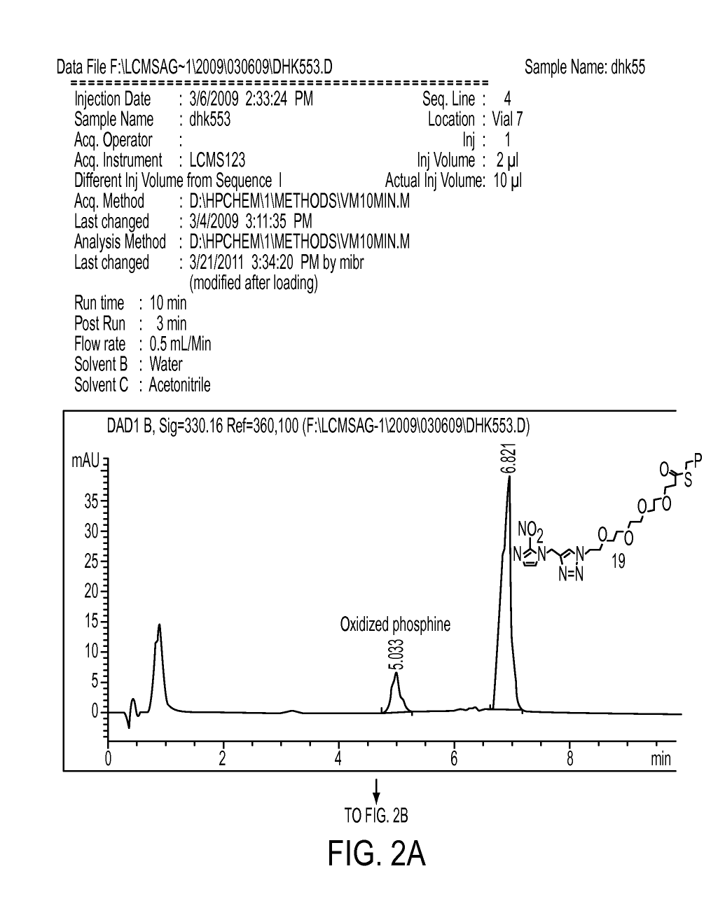

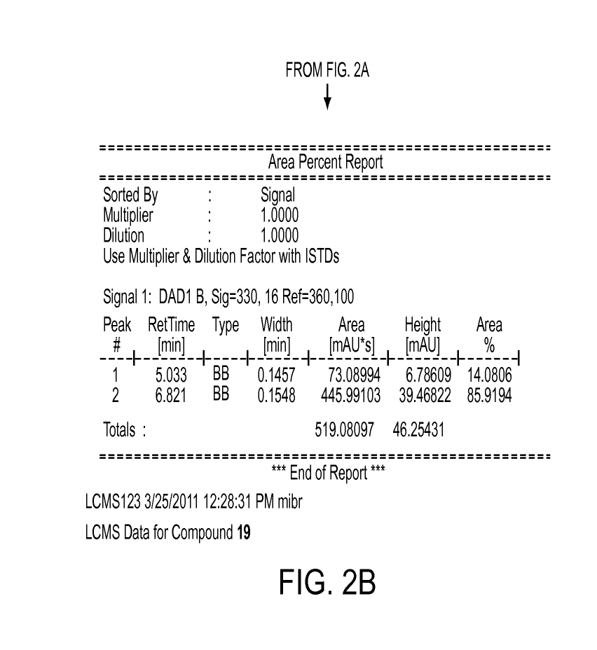

Phosphinemethane thiol ester 19 underwent 10% oxidation during the isolation using column chromatography (FIG. 2) at room temperature within an hour, while the arylphospine ester found to be stable under the same conditions. Also the diphenylphosphino methanethiol used for the synthesis of 19 is highly air sensitive unlike the diphenylphosphinephenol.

2. Application of Staudinger Ligation in Caspase 3 Imaging:

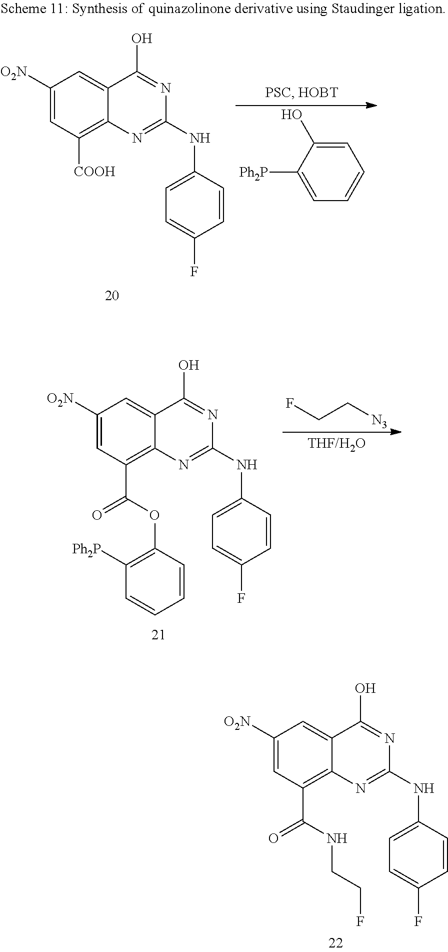

3. Application of Staudinger Ligation in Amino Acid Synthesis:

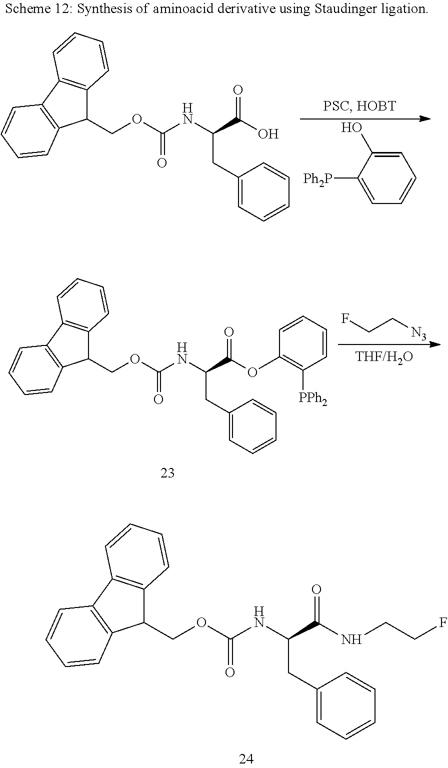

Experimentals:

All the substituted aryl phosphines were synthesized according to the general experimental procedure given below.

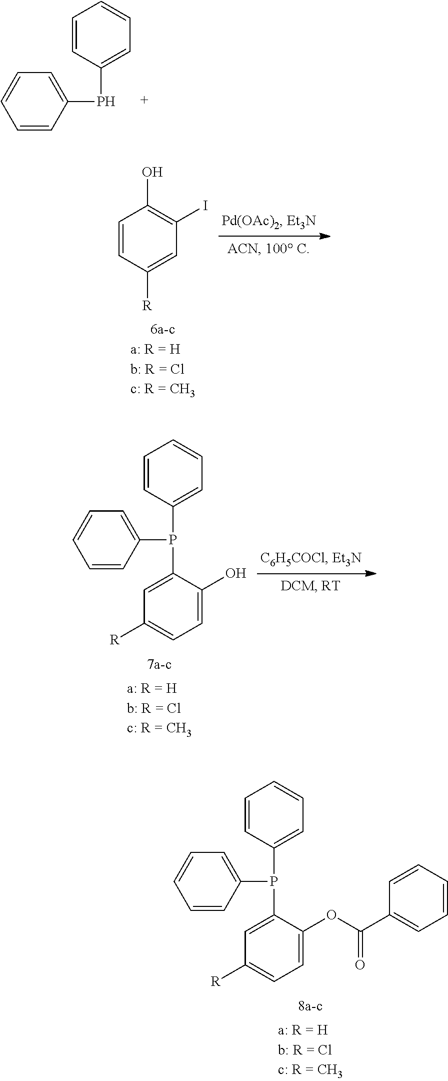

General Experimental Procedure for Phosphination:

To a round bottomed flask equipped with a magnetic stir bar, rubber septum, and argon inlet containing ACN (33 vol) was placed phenol (1 equiv). To this solution was added diphenylphosphine (1.2 equiv), Pd(OAc)₂ (0.05 equiv), triethylamine (6 equiv) and the reaction was allowed to stir at 100° C. for 15 h. The solvent was removed in vacuo. The residue was purified over silica gel using Hexanes:EtOAC as an eluent to afford the final product.

General Experimental Procedure for Benzoylation:

To a round bottomed flask equipped with a magnetic stir bar, rubber septum, and argon inlet containing DCM (100 vol) was placed diphenylphosphinophenol (1 equiv). To this solution was added benzoyl chloride (1.2 equiv), triethylamine (5 equiv) and the reaction was allowed to stir at room temperature for 15 h. The solvent was removed in vacuo. The residue was purified over silica gel using Hexanes:EtOAC as an eluent to afford the final product.

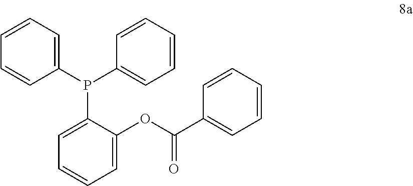 [SMILES: O=C(Oc1ccccc1P(c1ccccc1)c1ccccc1)c1ccccc1]

2-(diphenylphosphino)phenyl benzoate 8a:

¹H NMR (400 MHz, CDCl₃): δ 7.82 (d, J=7.2 Hz, 2H), 7.53 (t, J=7.6 Hz, 2H), 7.43-7.30 (m, 13H), 7.17 (dd, J=7.6, 6.8 Hz, 1H), 6.86-6.83 (m, 1H); MS (ESI, Pos.) m/z (M+H)⁺

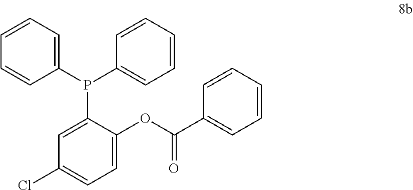 [SMILES: O=C(Oc1ccc(Cl)cc1P(c1ccccc1)c1ccccc1)c1ccccc1]

4-chloro-2-(diphenylphosphino)phenyl benzoate 8b:

¹H NMR (400 MHz, CDCl₃): δ 7.79 (dd, J=8.4, 1.2 Hz, 2H), 7.55-7.49 (m, 2H), 7.40-7.29 (m, 12H), 7.24-7.21 (m, 1H), 6.76 (dd, J=3.2, 2.4 Hz, 1H); MS (ESI, Pos.) m/z 417.0 (M+H)⁺

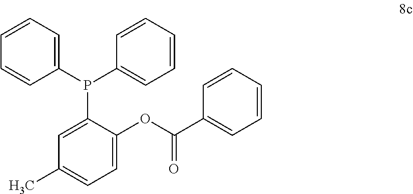 [SMILES: Cc1ccc(OC(=O)c2ccccc2)c(P(c2ccccc2)c2ccccc2)c1]

2-(diphenylphosphino)-4-methylphenyl benzoate 8c:

¹H NMR (400 MHz, CDCl₃): δ 7.81 (dd, J=8.4, 1.2 Hz, 2H). 7.53-7.49 (m, 1H), 7.35-7.29 (m, 12H), 7.22-7.14 (m, 2H), 6.64-6.62 (m, 1H) 2.22 (s, 3H); MS (ESI, Pos.) m/z 397.1 (M+H)⁺

General Experimental Procedure for the Synthesis of Heterocyclic Phosphines:

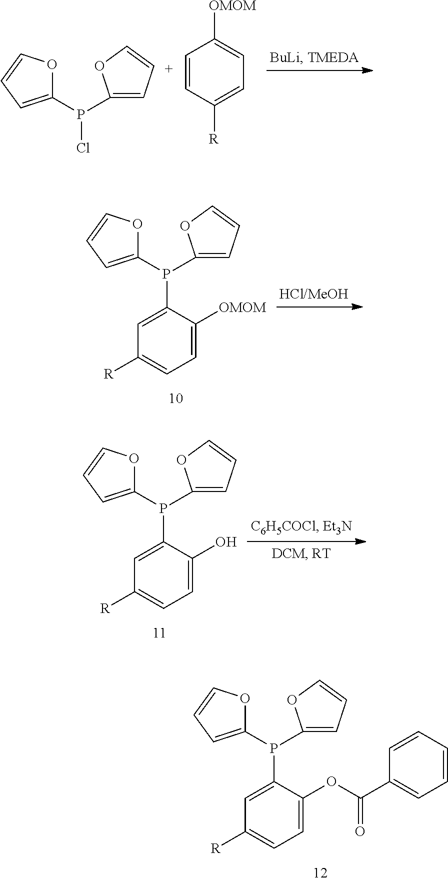

General Experimental Procedure for Phosphination:

To a round bottomed flask equipped with a magnetic stir bar, rubber septum, and argon inlet containing THF (10 vol) place protected phenol (1 equiv). To this solution add n-BuLi (1.2 equiv) at −78° C. and TMEDA (0.1 Equiv), and stir the reaction for 1 h. Add the chlorophosphine (1 equiv) in THF (5 vol) to the reaction mixture and stir at RT until the reaction is complete by LCMS. Quench the reaction mixture with water, extract with DCM, wash the organic layer with water and dry over Na₂SO₄. Remove the solvent in vacuo and purify the residue over silica gel using Hexanes:EtOAC as an eluent affords the final product.

General Experimental Procedure for Deprotection:

To a round bottomed flask equipped with a magnetic stir bar add the protected phosphine ester (1 equiv) in MeOH (5 vol). To this solution add 1N HCl in MeOH (1 vol) and stir the reaction at rt for 1 h. After the reaction is complete, evaporate the solvent in vacuo yields the phenol.

General Experimental Procedure for Benzoylation:

To a round bottomed flask equipped with a magnetic stir bar, rubber septum, and argon inlet containing DCM (100 vol) place phosphinophenol (1 equiv). To this solution add benzoyl chloride (1.2 equiv), triethylamine (5 equiv) and stir the reaction at room temperature for 15 h. Remove the solvent in vacuo and purify the residue over silica gel using Hexanes:EtOAC as an eluent affords the final product.

Synthesis of Imidazole Phosphine Ester:

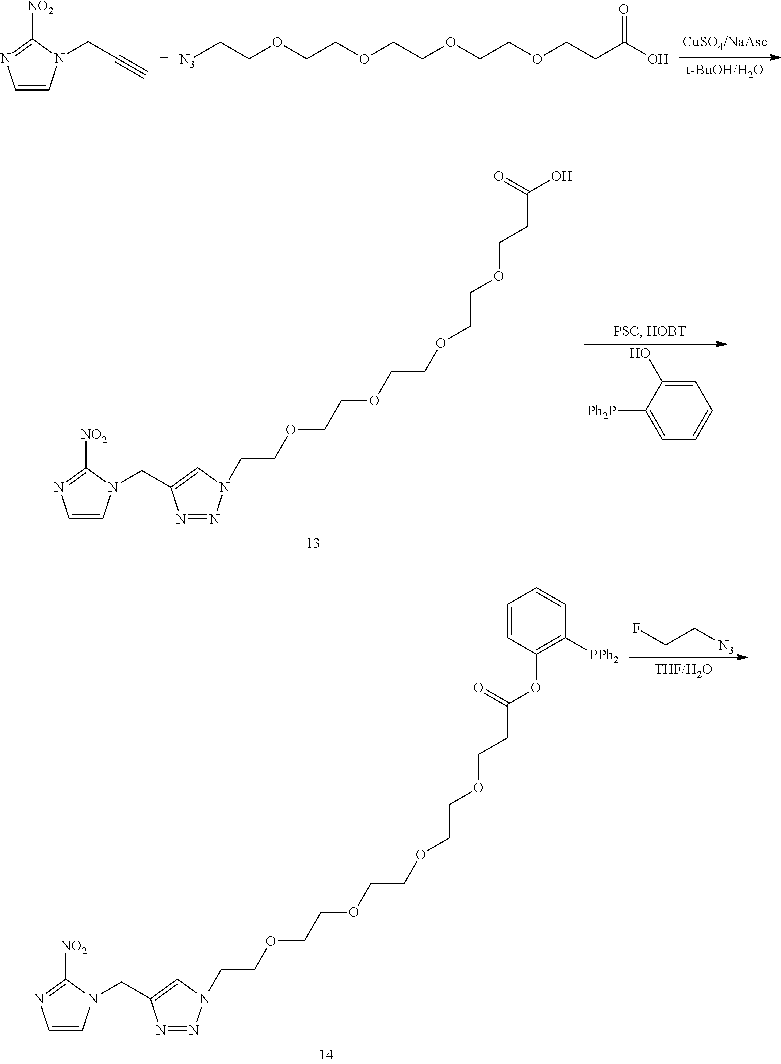

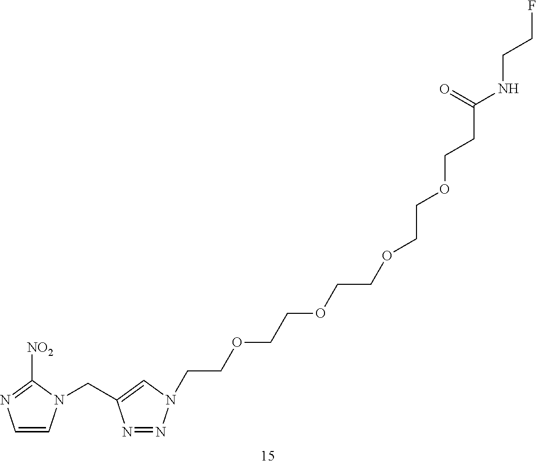 [SMILES: O=C(CCOCCOCCOCCOCCn1cc(Cn2ccnc2[N+](=O)[O-])nn1)NCCF]

Synthesis of Compound 13:

To a round bottomed flask equipped with a magnetic stir bar, rubber septum, and argon inlet containing t-BuOH: H₂0 (1:1, 4 ml) was placed pegylated azide (0.05 g, 0.17 mmol, 1 equiv). To this solution was added acetylene (0.027 g, 0.18 mmol, 1.05 equiv), CuSO₄.5H₂O (8.6 mg, 0.034 mmol, 0.2 equiv), sodium L-ascorbate (0.014, 0.069 mmol, 0.4 equiv) and the reaction was allowed to stir at room temperature for 3 h. After the reaction was complete, the reaction mixture was diluted with water and purified HPLC to give 0.06 g (80%) of the triazole 13 as white solid. MS (ESI, Pos.) m/z: 443.1 [M+H]⁺.

Synthesis of Compound 14:

A 5 mL microwave tube was charged with acid (0.02 g, 0.045 mmol, 1 equiv), PS-Carbodiimide (73 mg, 0.090 mmol, 2 equiv), 1-hydroxybenzotriazole (6.0 mg, 0.044 mmol, 0.99 equiv) and phenol (0.012 g, 0.045 mmol, 1 equiv) in dichloromethane (2 mL). The suspension was irradiated in an Emrys Optimizer microwave reactor (250 W) at 100° C. for 15 min. After cooling to room temperature the reaction mixture was diluted with MeOH/H₂O and purified by HPLC to yield the ester 14 (0.01 g, 33%).

Synthesis of Compound 15:

To a round bottomed flask equipped with a magnetic stir bar, rubber septum, and argon inlet containing THF: H₂0 (1:0.25, 2 ml) was placed phosphine ester (0.01 g, 0.014 mmol, 1 equiv). To this solution was added fluoroethylazide (excess) and the reaction was allowed to stir at 80° C. for 10 min. After the reaction was complete, the reaction mixture was diluted with water and purified HPLC to give 0.002 g (30%) of the amide 15 as white solid. MS (ESI, Pos.) m/z: 488.1 [M+H]⁺.

Synthesis of Imidazole Thiol Ester 19:

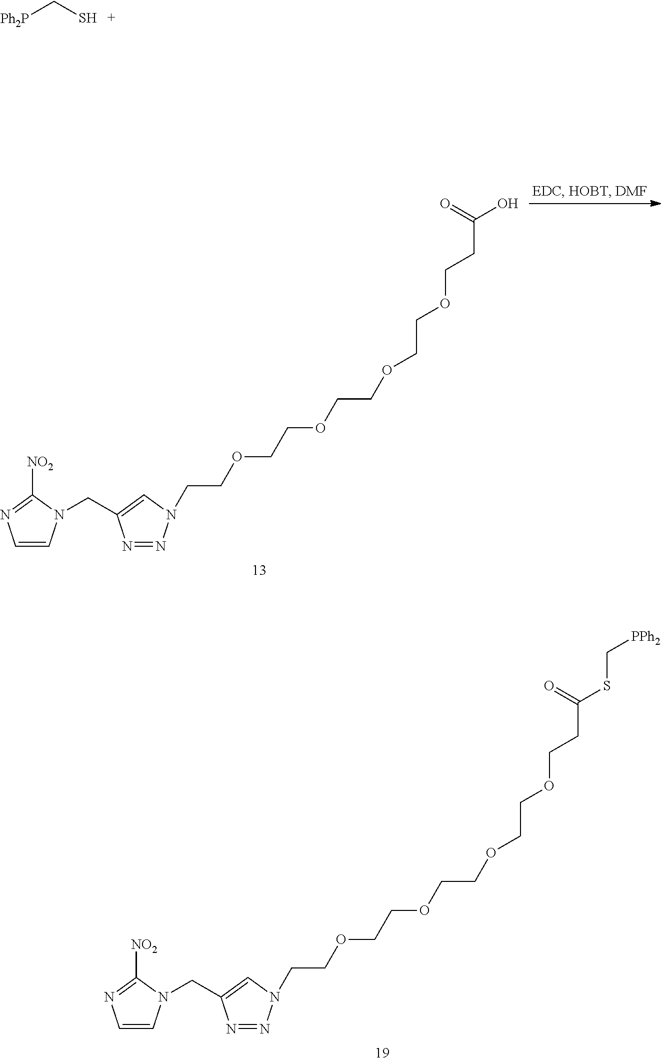

To a round bottomed flask equipped with a magnetic stir bar, rubber septum containing DMF (5 mL) was placed triazole acid (0.029 g, 0.052 mmol, 1 equiv). To this solution was added EDC (0.037 g, 0.19 mmol, 3 equiv), HOBt (0.026 g, 0.19 mmol, 3 equiv) and the reaction was allowed to stir at room temperature for 15 h. To this mixture thiol (0.15 g, 0.065 mmol, 1.5 equiv) was added and stirred at room temperature for 15 h. The solvent was removed in vacuo. The residue was purified over silica gel using Hexane:EtOAC (10:90) as an eluent to afford thiol ester 19 (0.01 g, 23%) as a white solid. MS: MS (ESI, Pos.) m/z: 657.1 [M+H]⁺. Oxidation of the phosphine thiol ester was observed during the isolation.

Synthesis of FETA:

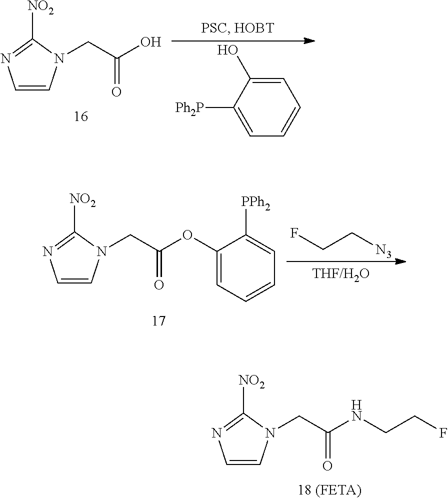

Synthesis of Compound 17:

A 5 mL microwave tube is charged with acid (0.05 g, 0.292 mmol, 1 equiv), PS-Carbodiimide (47 mg, 0.584 mmol, 2 equiv), 1-hydroxybenzotriazole (0.038 g, 0.29 mmol, 0.99 equiv) and phenol (0.081 g, 0.292 mmol, 1 equiv) in dichloromethane (1 mL) and DMF (1 mL). The suspension is irradiated in an Emrys Optimizer microwave reactor (250 W) at 100° C. for 15 min. After cooling to room temperature the reaction mixture is diluted with MeOH/H₂O and purification by HPLC affords the phosphine ester 17.

Synthesis of Compound 18:

To a round bottomed flask equipped with a magnetic stir bar, rubber septum, and argon inlet containing THF: H₂0 (1:0.25, 2 ml) add phosphine ester (0.01 g, 0.023 mmol, 1 equiv). To this solution add fluoroethylazide (excess) and stir the reaction at 80° C. for 10 min. After the reaction is complete, the reaction mixture is diluted with water and purified HPLC to give the amide 18.

Synthesis of Quinazolinone Amide:

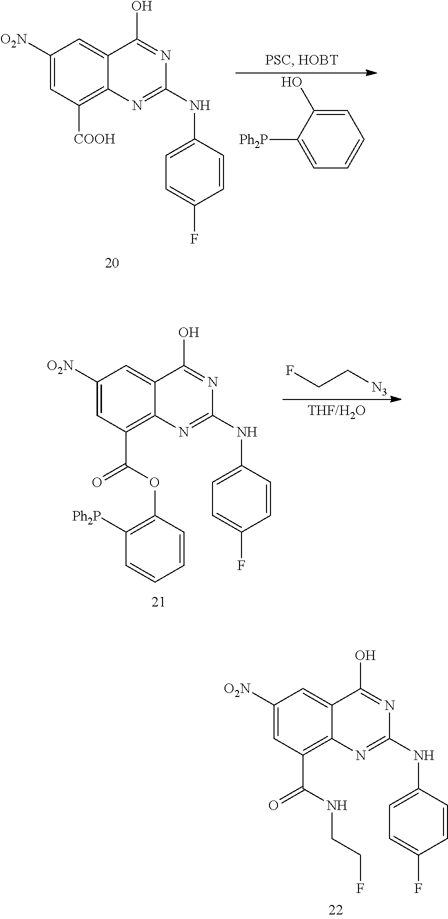

Synthesis of Compound 21:

A 5 mL microwave tube was charged with acid 20 (0.036 g, 0.105 mmol, 1 equiv), PS-Carbodiimide (17 mg, 0.209 mmol, 2 equiv), 1-hydroxybenzotriazole (0.013 g, 0.104 mmol, 0.99 equiv) and phenol (0.029 g, 0.105 mmol, 1 equiv) in dichloromethane (1 mL) and DMF (1 mL). The suspension was irradiated in an Emrys Optimizer microwave reactor (250 W) at 100° C. for 15 min. After cooling to room temperature the reaction mixture was diluted with MeOH/H₂O and filtered to yield the phosphine ester 21 as a yellow solid (0.05 g, 83%).

Synthesis of Compound 22:

To a round bottomed flask equipped with a magnetic stir bar, rubber septum, and argon inlet containing THF: H₂0 (1:0.25, 2 ml) add phosphine ester (1 equiv). To this solution add fluoroethylazide (excess) and stir the reaction at 80° C. for 10 min. After the reaction is complete, the reaction mixture is diluted with water and purified HPLC to give the amide 22.

Synthesis of Amino Acid Derivative:

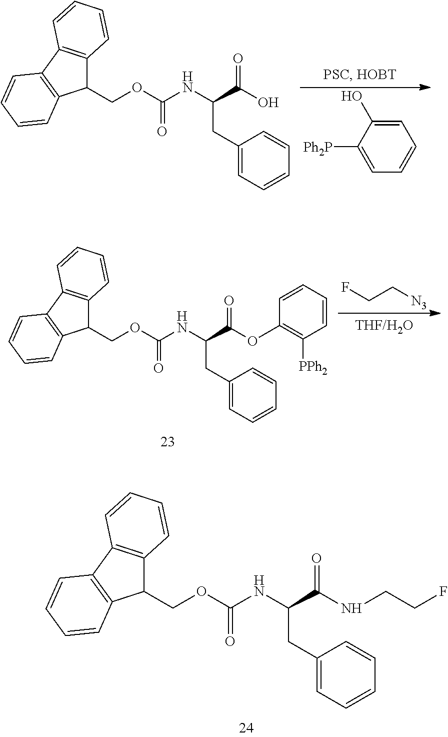

Synthesis of Compound 23:

A 5 mL microwave tube was charged with acid (0.115 g, 0.300 mmol, 1 equiv), PS-Carbodiimide (0.48 g, 0.593 mmol, 2 equiv), 1-hydroxybenzotriazole (0.039 g, 0.294 mmol, 0.99 equiv) and phenol (0.086 g, 0.311 mmol, 1 equiv) in dichloromethane (2 mL mL). The suspension was irradiated in an Emrys Optimizer microwave reactor (250 W) at 100° C. for 15 min. After cooling to room temperature the reaction mixture was diluted with MeOH/H₂O and purified by HPLC to yield the phosphine ester as white solid 23 (0.1 g, 53%).

Synthesis of Compound 24:

To a round bottomed flask equipped with a magnetic stir bar, rubber septum, and argon inlet containing THF: H₂0 (1:0.25, 2 ml) add phosphine ester (1 equiv). To this solution add fluoroethylazide (excess) and stir the reaction at 80° C. for 10 min. After the reaction is complete, the reaction mixture is diluted with water and purified HPLC to give the amide 24.

All the [F18] labeled amides were prepared using the general experimental procedure for Staudinger ligation as given below.

Synthesis of [F-18] Labeled Amide:

Aqueous [F-18]fluoride ion produced in the cyclotron target, is passed through an anion exchange resin cartridge. The [O-18]H₂O readily passes through the anion exchange resin while [F-18]fluoride is retained. The [F-18]fluoride is eluted from the column using either a solution of potassium carbonate (7.5 mg/mL of water)/Kryptofix® 222 (20 mg/mL of anhydrous acetonitrile or tetra butyl ammonium bicarbonate (0.6 mL) or tetra ethyl ammonium bicarbonate (5 mg/mL of water) is collected in a reaction vessel. The mixture is dried by heating between 70-115° C. under reduced pressure (250 mbar) and a stream of argon. This evaporation step removes the water and to produce anhydrous [F-18], which is much more reactive than aqueous [F-18]fluoride. A solution of the precursor, (˜5 mg) dissolved in THF or DMF or ACN or DMSO (0.5 mL) is added to the reaction vessel containing the anhydrous [F-18]Fluoride. The vessel is heated to approximately 80-150° C. for 3-15 min to induce displacement of the leaving group by [F-18]fluoride. After the reaction time, the [F-18]fluoro compound is either distilled or purified by semi-prep or ion-exchange or C-18 column in to a 5 mL vial containing phosphine ester in THF/H₂O (3:1, 0.5 mL) mixture.

If the F-18 fluorinated compound is purified by semi-prep or ion-exchange or C-18 column, then, depending on the mobile phase/solvent combination used, it is reformulated either with THF/H2O or ACN/H2O (3:1, 0.5 mL) mixture before adding to the phosphine ester mixture.

This mixture is heated in an oil bath or 2^{nd} reaction pot for 5-20 min at 80-100° C. The crude mixture is purified by semi-prep HPLC using appropriate mobile phase. Appropriate mobile phases for semi-preparative reverse phase HPLC include aqueous acetonitrile or methanol with an optional additive such as formic acid. After collection of the purified material, the product can either be used without reformulation or can be reformulated by diluting with water (20-50 mL), passing through C-18 and the mixture is collected onto a C-18 cartridge. The cartridge is rinsed with water (10 mL) and the product is eluted with EtOH (0.5-1.0 mL) into a vial with or without stabilizer and diluted with either 0.9% saline or water (4.5-9.0 mL).

While this invention has been described in conjunction with the specific embodiments outlined above, it is evident that many alternatives, modifications, and variations will be apparent to those skilled in the art. Accordingly, the preferred embodiments of the invention as set forth above are intended to be illustrative, not limiting. Various changes may be made without departing from the spirit and scope of the inventions as defined in the following claims.

What is claimed is:

1. A method for generating a radiolabeled tracer, the method comprising:
providing a compound of the following Formula I: 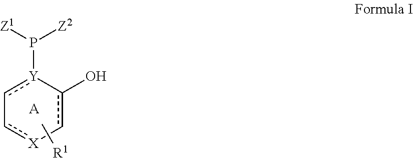
wherein X is C, N, or a bond;
wherein Y is C or N;
wherein:
ring A is either aromatic or saturated when X is C;
ring A is aromatic when X is N; and
ring A is saturated when X is a bond;
wherein R¹ is selected from the group consisting of H, an electron withdrawing group, and an electron donating group;
wherein:
Z¹ and Z², together with the phosphorus atom to which they are attached to, form a substituted or unsubstituted, heterocyclic ring;
Z¹ and Z² together are ferrocene, with the P atom being connected to each cyclopentadiene ring; or
Z¹ and Z² are each independently selected from the group consisting of carbocyclic, heterocyclic, aryl, heteroaryl, NR²R³;
wherein R² and R³ are each independently selected from the group consisting of H, alkyl, aryl, an electron withdrawing group, and an electron donating group; and
wherein, when R¹ is H, at least one of Z¹ and Z² is an aryl which includes a substituent selected from the group consisting of an electron withdrawing group and an electron donating group;
a first step of condensing the OH of Formula I with an acid to produce a phosphine ester; and
a second step of performing Staudinger ligation to generate the radiolabeled tracer by treating the phosphine ester produced in the first step with a radiolabeled azide having a PET radioisotope moiety Q as the radiolabel.

2. The method of claim 1;
wherein the electron withdrawing group is selected from the group consisting of CN, CF₃, F, Cl, Br, COR⁴, CONH₂, SONH₂, and SO₃R⁵;
wherein the electron donating group is selected from the group consisting of alkyl, aryl, O-alkyl, O-aryl, NH₂, NHR⁶, NR⁷R⁸, and NHCOMe; and
wherein R⁴, R⁵, R⁶, R⁷, and R⁸ are each independently selected from the group consisting of H, alkyl, and aryl.

3. The method of claim 1;
wherein the acid used in the first step is of the general formula:
R′—COOH
where R′ is selected from the group consisting of alkyl, aryl, aminoalkyl, sugar, heterocycle, and heteroaryl.

4. The method of claim 3;
wherein the radiolabeled azide used in the second step is of the general formula:
Q-B—N₃;
where B is selected from the group consisting of alkyl, aryl, aminoalkyl, sugar, heterocycle, and heteroaryl; and
wherein the radiolabeled tracer generated by the second step is of the general formula:
Q-B—NH—CO—R′.

5. The method of claim 1;
wherein the Q is selected from the group consisting of ¹¹C, ¹³N, ¹⁵O, and ¹⁸F.

6. The method of claim 1;
wherein at least one of Z¹ and Z² is selected from the group consisting of cyclohexyl, cyclopentyl, phenyl, 4-R⁹-substituted aryl, 2-naphthyl, NR²R³, N-pyrrolidinyl, N-morpholinyl, pyrrolyl, 4-pyridyl, cyclopentadiene, mono-hetero substituted five-member aromatic ring, and di-hetero substituted five-member aromatic ring; and
wherein R⁹ is selected from the group consisting of H, alkyl, aryl, an electron withdrawing group, and an electron donating group.

7. The method of claim 1;
wherein Z¹ and Z², together with the phosphorus atom to which they are attached to, form a substituted or unsubstituted, heterocyclic ring;
wherein the heteroatoms are connected directly to P; and
Z¹ and Z² together is selected from the group consisting of:
1,2 dihydroxybenzene, where the two O atoms are directly connected to the P atom to form a five-membered ring;
ethylenediamine and its N-substituted derivatives, where the two N atoms are directly connected to the P atom to form a five-membered ring; and
ethylene glycol and its C-substituted derivatives, where the two O atoms are directly connected to the P atom to form a five-membered ring.

8. The method of claim 1;
wherein the compound of Formula I takes the form of: 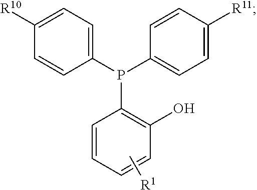
wherein R¹⁰ and R¹¹ are each independently selected from the group consisting of H, alkyl, aryl, an electron withdrawing group, and an electron donating group;
wherein the acid used in the first step is of the general formula:
R′—COOH
where R′ is selected from the group consisting of alkyl, aryl, aminoalkyl, sugar, heterocycle, and heteroaryl;
wherein the radiolabeled azide used in the second step is of the general formula:
Q-B—N₃;
where B is selected from the group consisting of alkyl, aryl, aminoalkyl, sugar, heterocycle, and heteroaryl;
wherein the radiolabeled tracer generated by the second step is of the general formula:
Q-B—NH—CO—R′; and
wherein the Q is selected from the group consisting of ¹¹C, ¹³N, ¹⁵O, and ¹⁸F.

9. The method of claim 1;
wherein the compound of Formula I takes the form of: 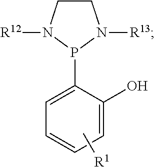
wherein R¹² and R¹³ are each independently selected from the group consisting of H, alkyl, aryl, an electron withdrawing group, and an electron donating group;
wherein the acid used in the first step is of the general formula:
R′—COOH
where R′ is selected from the group consisting of alkyl, aryl, aminoalkyl, sugar, heterocycle, and heteroaryl;
wherein the radiolabeled azide used in the second step is of the general formula:
Q-B—N₃;
where B is selected from the group consisting of alkyl, aryl, aminoalkyl, sugar, heterocycle, and heteroaryl;
wherein the radiolabeled tracer generated by the second step is of the general formula:
Q-B—NH—CO—R′; and
wherein the Q is selected from the group consisting of ¹¹C, ¹³N, ¹⁵O, and ¹⁸F.

10. The method of claim 1;
wherein the compound of Formula I takes the form of: 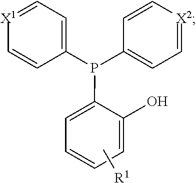
wherein X¹ and X² are each independently selected from the group consisting of CH and N;
wherein the acid used in the first step is of the general formula:
R′—COOH
where R′ is selected from the group consisting of alkyl, aryl, aminoalkyl, sugar, heterocycle, and heteroaryl;
wherein the radiolabeled azide used in the second step is of the general formula:
Q-B—N₃;
where B is selected from the group consisting of alkyl, aryl, aminoalkyl, sugar, heterocycle, and heteroaryl;
wherein the radiolabeled tracer generated by the second step is of the general formula:
Q-B—NH—CO—R′; and
wherein the Q is selected from the group consisting of ¹¹C, ¹³N, ¹⁵O, and ¹⁸F.

11. The method of claim 1;
wherein the compound of Formula I takes the form of: 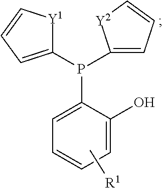
wherein Y¹ and Y² are each independently selected from the group consisting of S, O, NH, and CH₂;
wherein the acid used in the first step is of the general formula:
R′—COOH
where R′ is selected from the group consisting of alkyl, aryl, aminoalkyl, sugar, heterocycle, and heteroaryl;
wherein the radiolabeled azide used in the second step is of the general formula:
Q-B—N₃;
where B is selected from the group consisting of alkyl, aryl, aminoalkyl, sugar, heterocycle, and heteroaryl;
wherein the radiolabeled tracer generated by the second step is of the general formula:
Q-B—NH—CO—R′; and
wherein the Q is selected from the group consisting of ¹¹C, ¹³N, ¹⁵O, and ¹⁸F.

12. The method of claim 1;
wherein the compound of Formula I takes the form of: 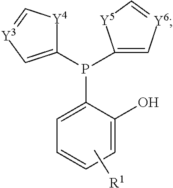
wherein Y³, Y⁴, Y⁵, and Y⁶ are each independently selected from the group consisting of S, O, NH, and CH₂; and
wherein Y³, Y⁴, Y⁵, and Y⁶ are each independently selected from the group consisting of S, O, NH, and CH₂;
wherein the acid used in the first step is of the general formula:
R′—COOH;
where R′ is selected from the group consisting of alkyl, aryl, aminoalkyl, sugar, heterocycle, and heteroaryl;
wherein the radiolabeled azide used in the second step is of the general formula:
Q-B—N₃;
where B is selected from the group consisting of alkyl, aryl, aminoalkyl, sugar, heterocycle, and heteroaryl;
wherein the radiolabeled tracer generated by the second step is of the general formula:
Q-B—NH—CO—R′; and
wherein the Q is selected from the group consisting of ¹¹C, ¹³N, ¹⁵O, and ¹⁸F.

13. The method of claim 1;
wherein the compound of Formula I takes the form of: 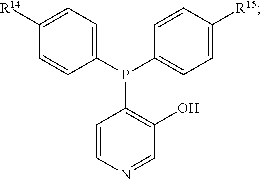
wherein R¹⁴ and R¹⁵ are each independently selected from the group consisting of H, alkyl, aryl, an electron withdrawing group, and an electron donating group;
wherein the acid used in the first step is of the general formula:
R′—COOH
where R′ is selected from the group consisting of alkyl, aryl, aminoalkyl, sugar, heterocycle, and heteroaryl;
wherein the radiolabeled azide used in the second step is of the general formula:
Q-B—N₃;
where B is selected from the group consisting of alkyl, aryl, aminoalkyl, sugar, heterocycle, and heteroaryl;
wherein the radiolabeled tracer generated by the second step is of the general formula:
Q-B—NH—CO—R′; and
wherein the Q is selected from the group consisting of ¹¹C, ¹³N, ¹⁵O, ¹⁸F.

14. The method of claim 1;
wherein the compound of Formula I takes the form of: 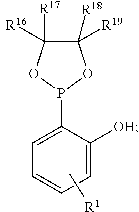
wherein R¹⁶ to R¹⁹ are each independently selected from the group consisting of H, alkyl, aryl, an electron withdrawing group, and an electron donating group;
wherein the acid used in the first step is of the general formula:
R′—COOH
where R′ is selected from the group consisting of alkyl, aryl, aminoalkyl, sugar, heterocycle, and heteroaryl;
wherein the radiolabeled azide used in the second step is of the general formula:
Q-B—N₃;
where B is selected from the group consisting of alkyl, aryl, aminoalkyl, sugar, heterocycle, and heteroaryl;
wherein the radiolabeled tracer generated by the second step is of the general formula:
Q-B—NH—CO—R′; and
wherein the Q is selected from the group consisting of ¹¹C, ¹³N, ¹⁵O, and ¹⁸F.

15. The method of claim 1;
wherein the compound of Formula I takes the form of: 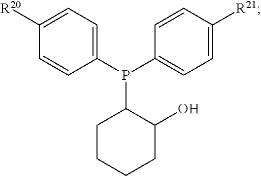
wherein R²⁰ and R²¹ are each independently selected from the group consisting of H, alkyl, aryl, an electron withdrawing group, and an electron donating group;
wherein the acid used in the first step is of the general formula:
R′—COOH
where R′ is selected from the group consisting of alkyl, aryl, aminoalkyl, sugar, heterocycle, and heteroaryl;
wherein the radiolabeled azide used in the second step is of the general formula:
Q-B—N₃;
where B is selected from the group consisting of alkyl, aryl, aminoalkyl, sugar, heterocycle, and heteroaryl;
wherein the radiolabeled tracer generated by the second step is of the general formula:
Q-B—NH—CO—R′; and
wherein the Q is selected from the group consisting of ¹¹C, ¹³N, ¹⁵O, and ¹⁸F.

16. The method of claim 1;
wherein the compound of Formula I takes the form of: 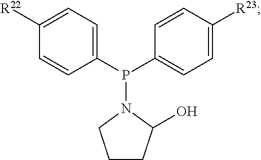
wherein R²² and R²³ are each independently selected from the group consisting of H, alkyl, aryl, an electron withdrawing group, and an electron donating group;
wherein the acid used in the first step is of the general formula:
R′—COOH
where R′ is selected from the group consisting of alkyl, aryl, aminoalkyl, sugar, heterocycle, and heteroaryl;
wherein the radiolabeled azide used in the second step is of the general formula:
Q-B—N₃;
where B is selected from the group consisting of alkyl, aryl, aminoalkyl, sugar, heterocycle, and heteroaryl;
wherein the radiolabeled tracer generated by the second step is of the general formula:
Q-B—NH—CO—R′; and
wherein the Q is selected from the group consisting of ¹¹C, ¹³N, ¹⁵O, and ¹⁸F.

17. The method of claim 1;
wherein X is C;
wherein Y is C;
wherein ring A is aromatic;
wherein Z¹ and Z² are both cyclohexane;
wherein the acid used in the first step is of the general formula:
R′—COOH
where R′ is selected from the group consisting of alkyl, aryl, aminoalkyl, sugar, heterocycle, and heteroaryl;
wherein the radiolabeled azide used in the second step is of the general formula:
Q-B—N₃;
where B is selected from the group consisting of alkyl, aryl, aminoalkyl, sugar, heterocycle, and heteroaryl;
wherein the radiolabeled tracer generated by the second step is of the general formula:
Q-B—NH—CO—R′; and
wherein the Q is selected from the group consisting of ¹¹C, ¹³N, ¹⁵O, and ¹⁸F.

18. The method of claim 1;
wherein X is C;
wherein Y is C;
wherein ring A is aromatic;
wherein Z¹ and Z² are both cyclopentane;
wherein the acid used in the first step is of the general formula:
R′—COOH
where R′ is selected from the group consisting of alkyl, aryl, aminoalkyl, sugar, heterocycle, and heteroaryl;
wherein the radiolabeled azide used in the second step is of the general formula:
Q-B—N₃;
where B is selected from the group consisting of alkyl, aryl, aminoalkyl, sugar, heterocycle, and heteroaryl;
wherein the radiolabeled tracer generated by the second step is of the general formula:
Q-B—NH—CO—R′; and
wherein the Q is selected from the group consisting of ¹¹C, ¹³N, ¹⁵O, and ¹⁸F.

19. The method of claim 1;
wherein X is C;
wherein Y is C;
wherein ring A is aromatic;
wherein Z¹ and Z² are both naphthalene;
wherein the acid used in the first step is of the general formula:
R′—COOH
where R′ is selected from the group consisting of alkyl, aryl, aminoalkyl, sugar, heterocycle, and heteroaryl;
wherein the radiolabeled azide used in the second step is of the general formula:
Q-B—N₃;
where B is selected from the group consisting of alkyl, aryl, aminoalkyl, sugar, heterocycle, and heteroaryl;
wherein the radiolabeled tracer generated by the second step is of the general formula:
Q-B—NH—CO—R′; and
wherein the Q is selected from the group consisting of ¹¹C, ¹³N, ¹⁵O, and ¹⁸F.

20. The method of claim 1;
wherein X is C;
wherein Y is C;
wherein ring A is aromatic;
wherein Z¹ and Z² are both NR²R³;
wherein the acid used in the first step is of the general formula:
R′—COOH
where R′ is selected from the group consisting of alkyl, aryl, aminoalkyl, sugar, heterocycle, and heteroaryl;
wherein the radiolabeled azide used in the second step is of the general formula:
Q-B—N₃;
where B is selected from the group consisting of alkyl, aryl, aminoalkyl, sugar, heterocycle, and heteroaryl;
wherein the radiolabeled tracer generated by the second step is of the general formula:
Q-B—NH—CO—R′; and
wherein the Q is selected from the group consisting of ¹¹C, ¹³N, ¹⁵O, and ¹⁸F.

21. The method of claim 1;
wherein X is C;
wherein Y is C;
wherein ring A is aromatic;
wherein Z¹ and Z² are both N-pyrrolidine;
wherein the acid used in the first step is of the general formula:
R′—COOH
where R′ is selected from the group consisting of alkyl, aryl, aminoalkyl, sugar, heterocycle, and heteroaryl;
wherein the radiolabeled azide used in the second step is of the general formula:
Q-B—N₃;
where B is selected from the group consisting of alkyl, aryl, aminoalkyl, sugar, heterocycle, and heteroaryl;
wherein the radiolabeled tracer generated by the second step is of the general formula:
Q-B—NH—CO—R′; and
wherein the Q is selected from the group consisting of ¹¹C, ¹³N, ¹⁵O, and ¹⁸F.

22. The method of claim 1;
wherein X is C;
wherein Y is C;
wherein ring A is aromatic;
wherein Z¹ and Z² are both N-morpholine;
wherein the acid used in the first step is of the general formula:
R′—COOH
where R′ is selected from the group consisting of alkyl, aryl, aminoalkyl, sugar, heterocycle, and heteroaryl;
wherein the radiolabeled azide used in the second step is of the general formula:
Q-B—N₃;
where B is selected from the group consisting of alkyl, aryl, aminoalkyl, sugar, heterocycle, and heteroaryl;
wherein the radiolabeled tracer generated by the second step is of the general formula:
Q-B—NH—CO—R′; and
wherein the Q is selected from the group consisting of ¹¹C, ¹³N, ¹⁵O, and ¹⁸F.

23. The method of claim 1;
wherein X is C;
wherein Y is C;
wherein ring A is aromatic;
wherein Z¹ and Z² are both N-pyrrole;
wherein the acid used in the first step is of the general formula:
R′—COOH
where R′ is selected from the group consisting of alkyl, aryl, aminoalkyl, sugar, heterocycle, and heteroaryl;
wherein the radiolabeled azide used in the second step is of the general formula:
Q-B—N₃;
where B is selected from the group consisting of alkyl, aryl, aminoalkyl, sugar, heterocycle, and heteroaryl;
wherein the radiolabeled tracer generated by the second step is of the general formula:
Q-B—NH—CO—R′; and
wherein the Q is selected from the group consisting of ¹¹C, ¹³N, ¹⁵O, and ¹⁸F.

24. The method of claim 1;
wherein X is C;
wherein Y is C;
wherein ring A is aromatic; and
wherein Z¹ and Z², together with the phosphorus atom to which they are attached to, form a substituted or unsubstituted, heterocyclic ring;
wherein the heteroatoms are connected directly to P; and
wherein Z¹ and Z² together are 1,2 dihydroxybenzene, where the two O atoms are directly connected to the P atom to form a five-membered ring.

25. The method of claim 1;
wherein X is C;
wherein Y is C;
wherein ring A is aromatic;
wherein Z¹ and Z² together are ferrocene, with the P atom being connected to each cyclopentadiene ring;
wherein the acid used in the first step is of the general formula:
R′—COOH
where R′ is selected from the group consisting of alkyl, aryl, aminoalkyl, sugar, heterocycle, and heteroaryl;
wherein the radiolabeled azide used in the second step is of the general formula:
Q-B—N₃;
where B is selected from the group consisting of alkyl, aryl, aminoalkyl, sugar, heterocycle, and heteroaryl;
wherein the radiolabeled tracer generated by the second step is of the general formula:
Q-B—NH—CO—R′; and
wherein the Q is selected from the group consisting of ¹¹C, ¹³N, ¹⁵O, and ¹⁸F.
